(19) United States
(12) Patent Application Publication
(10) Pub. No.: US 2026/0021054 A1
(43) Pub. Date: Jan. 22, 2026

(54) METHODS AND COMPOSITIONS FOR TREATING PARASITIC DISEASES USING NANOBUBBLES AND ANTI-PARASITIC AGENTS

(71) Applicant: Moleaer, Inc., Hawthorne, CA (US)

(72) Inventors: Warren Stuart Russell, Hawthorne, CA (US); Sohail Akhter, Carson, CA (US)

(21) Appl. No.: 19/265,699

(22) Filed: Jul. 10, 2025

Related U.S. Application Data
(60) Provisional application No. 63/672,508, filed on Jul. 17, 2024.

(51) Int. Cl. A61K 9/51; A01K 61/13; A61K 45/06

(52) U.S. Cl. CPC A61K 9/51; A01K 61/13; A61K 45/06

(57) ABSTRACT

Provided herein are methods and compositions for treating parasitic diseases using nanobubbles in combination with at least one anti-parasitic agent.

### CLAIM OF PRIORITY

This application claims priority to U.S. Provisional Application Ser. No. 63/672,508, filed on Jul. 17, 2025, the entire contents of which are hereby incorporated by reference.

### TECHNICAL FIELD

This disclosure relates to methods and compositions for treating parasitic diseases.

### BACKGROUND

In fish farming, parasitic diseases are a significant problem because they interfere with stable production. Among parasitic diseases, monogeneans belonging to the phylum Monogenea or Caligus of the phylum Arthropoda are considered one of the biggest problems because they occur in many cultured fish. Monogeneans are commonly referred to as skin parasites and gill parasites. Skin parasites, such as Benedenia girellae and Neobenedenia seriolae, are known to parasitize many fish species, including tang, red snapper, halibut, flounder, and others. These parasites cause symptoms like redness of the epidermis, friction of the fins, clouding of the eyeballs, and excessive mucus secretion. Abnormal swimming behaviors, such as rubbing the body against the cage, are often observed, leading to worsened symptoms and increased infection risk.

In Chile, parasitosis of farmed salmon caused by Caligus rogercresseyi is treated with antiparasitics administered orally and through immersion baths. Commonly used immersion antiparasitics include organophosphates (azamethiphos), pyrethroids (deltamethrin and cypermethrin), benzoylurea (hexaflumuron), and hydrogen peroxide. These treatments are complex and subject to logistical challenges, with effectiveness varying between centers and over time. Factors influencing treatment success include parasite sensitivity, treatment application, and environmental conditions.

Gill parasites, such as Heteraxine heterocerca, Zeuxapta japonica, and Microcotyle sebastis, cause discoloration of gills, anemia, and reduced body condition. Similar to skin parasites, fish exhibit abnormal swimming behaviors, increasing the risk of pathogen infection and damage. Traditional treatment methods, including freshwater baths or high-concentration hydrogen peroxide baths, are labor-intensive and stressful for the fish.

### SUMMARY

The inventors have discovered that using nanobubbles in conjunction with antiparasitic agents enhances the removal of parasites. The unique properties of nanobubbles increase the efficacy of these treatments, leading to more complete control of ectoparasites. In particular, nanobubbles improve the distribution of antiparasitic agents, enhancing contact with ectoparasites. In addition, nanobubbles increase the solubility and stability of antiparasitic agents, ensuring a consistent therapeutic effect. The small size and surface charge of nanobubbles also help disperse the antiparasitic solution uniformly, increasing treatment coverage and efficiency.

Accordingly, aspects of the present disclosure provide a method that includes preparing a therapeutic composition comprising nanobubbles and at least one anti-parasitic agent in a liquid carrier, and topically applying the therapeutic composition to a body suffering from a parasitic disease.

In some embodiments, the concentration of nanobubbles in the therapeutic composition is at least 10⁶ nanobubbles per cm³, at least 10⁷ nanobubbles per cm³, at least 10⁸ nanobubbles per cm³, at least 10⁹ nanobubbles per cm³, at least 10¹⁰ nanobubbles per cm³, or at least 10¹¹ nanobubbles per cm³.

In some embodiments, the antiparasitic agent is selected from the group consisting of hydrogen peroxide, organophosphates, pyrethroids, benzylureas, potassium ions, and combinations thereof.

In some embodiments, the body suffering from a parasitic disease is a marine fish and the method includes applying the therapeutic composition to the skin, gills, or both of the marine fish. In some embodiments, the marine fish is a species from the order Perciformes.

In some embodiments, the parasitic disease includes ectoparasites selected from the group consisting of monogeneans, crustacean copepods, and combinations thereof.

In some embodiments, topically applying the therapeutic composition includes immersing the body (e.g., a fish) in the therapeutic composition. The body (e.g., a fish) may be included in a cage and immersed with the therapeutic composition in the cage. In other embodiments, a perforated hose is used to topically apply the therapeutic composition to the body (e.g., a fish).

Also disclosed is a therapeutic composition that includes nanobubbles and at least one anti-parasitic agent in a liquid carrier.

In some embodiments, the concentration of nanobubbles in the therapeutic composition is at least 10⁶ nanobubbles per cm³, at least 10⁷ nanobubbles per cm³, at least 10⁸ nanobubbles per cm³, at least 10⁹ nanobubbles per cm³, at least 10¹⁰ nanobubbles per cm³, or at least 10¹¹ nanobubbles per cm³.

In some embodiments, the antiparasitic agent is selected from the group consisting of hydrogen peroxide, organophosphates, pyrethroids, benzylureas, potassium ions, and combinations thereof.

Other features and advantages of the invention will be apparent from the following detailed description, and from the claims.

### DETAILED DESCRIPTION

Methods and compositions described herein involve treating parasitic diseases with topically applied therapeutic compositions comprising nanobubbles combined with at least one anti-parasitic agent. As used herein, the term “nanobubble” refers to a bubble that has a diameter of less than one micron. Nanobubbles have several unique properties such as high gas solubility into the liquid due to their high internal pressure and long lifetime in liquid due to their negatively charged surfaces.

The therapeutic composition may be used to treat any type of body suffering from a parasitic disease. For example, the therapeutic composition may be used to treat marine fish, including those from the order Perciformes, such as salmon, trout, bream, tilapia, and yellowtail. Examples of parasites that can be treated include monogeneans and crustacean copepods, such as Neobenedenia girellae, Benedenia seriolae, and Caligus spp.

According to methods and compositions described herein, nanobubbles are combined with one or more anti-parasitic agents to form a therapeutic composition in the form of a treatment bath. Non-limiting examples of anti-parasitic agents include hydrogen peroxide, organophosphates, pyrethroids, and benzoylureas. The resulting treatment bath is then topically applied to the body suffering from parasitic disease. For example, in the case of fish, the composition is topically applied to the skin and/or gills of the fish.

Any method or apparatus known in the art or described herein can be used to generate nanobubbles in methods and compositions provided herein. Non-limiting examples of methods and apparatuses for generating nanobubbles that can be used in methods and systems described herein are provided in U.S. Pat. Nos. 10,591,231 and 11,331,633, the entire contents of which are herein incorporated by reference for the purposes and subject matter referenced herein.

A nanobubble generator for use in methods and compositions described herein is capable of generating a high concentration of nanobubbles. In some embodiments, the nanobubble generator can generate nanobubbles at a concentration of at least 10⁶ nanobubbles per cm³. In some embodiments, the nanobubble concentration is at least 10⁷ nanobubbles per cm³, at least 10⁸ nanobubbles per cm³, at least 10⁹ nanobubbles per cm³, at least 10¹⁰ nanobubbles per cm³, or at least 10¹¹ nanobubbles per cm³.

The nanobubble concentration is expressed as nanobubbles per cm³. It is measured by collecting 3 samples from the nanobubble generator and analyzing each sample within 20 minutes after it has been obtained by Nanoparticle Tracking Analysis using a Nanosight NS3000 analyzer available from Malvern PANalytical. Each sample is filtered using a 0.45 m filter before it is analyzed using the Nanosight NS3000 analyzer.

Any gas can be used to generate nanobubbles. Non-limiting examples of gases that can be used to generate nanobubbles include air, hydrogen, biogas, methane, carbon dioxide, nitrogen, argon or other inert gases, oxygen, or ozone.

To prepare the therapeutic composition, nanobubbles in a liquid carrier, e.g., water, are combined with a solution or dispersion featuring one or more anti-parasitic agents in a liquid bath, e.g., a water bath. In some embodiments, this preparation may include creating multiple mixtures utilizing distinct containers and varying volumes of liquid. The body being treated may then be immersed directly in the therapeutic composition. Alternatively, the body being treated may be placed within a cage filled with a liquid such as a seawater, after which the therapeutic composition is introduced into the cage. In some embodiments, the initially formed therapeutic composition is injected into a large flow of oxygenated nanobubble water to form a second therapeutic composition that is conveyed and distributed, e.g., through one or more perforated hoses into the immersion bath or cage.

Particular embodiments of the subject matter have been described. Other embodiments are within the scope of the following claims.

What is claimed is:

1. A method comprising:
preparing a therapeutic composition comprising nanobubbles and at least one anti-parasitic agent in a liquid carrier; and
topically applying the therapeutic composition to a body suffering from a parasitic disease.

2. The method of claim 1, wherein the concentration of nanobubbles in the therapeutic composition is at least 10⁶ nanobubbles per cm³, at least 10⁷ nanobubbles per cm³, at least 10⁸ nanobubbles per cm³, at least 10⁹ nanobubbles per cm³, at least 10¹⁰ nanobubbles per cm³, or at least 10¹¹ nanobubbles per cm³.

3. The method of claim 1, wherein the antiparasitic agent is selected from the group consisting of hydrogen peroxide, organophosphates, pyrethroids, benzylureas, potassium ions, and combinations thereof.

4. The method of claim 1, wherein the body suffering from a parasitic disease is a marine fish and the method comprises applying the therapeutic composition to the skin, gills, or both of the marine fish.

5. The method of claim 4, wherein the marine fish is a species from the order Perciformes.

6. The method of claim 1, wherein the parasitic disease comprises ectoparasites selected from the group consisting of monogeneans, crustacean copepods, and combinations thereof.

7. The method of claim 1, wherein topically applying the therapeutic composition comprises immersing the body in the therapeutic composition.

8. The method of claim 7, wherein the body is included in a cage and immersed with the therapeutic composition in the cage.

9. The method of claim 1, comprising using a perforated hose to topically apply the therapeutic composition to the body.

10. A therapeutic composition comprising nanobubbles and at least one anti-parasitic agent in a liquid carrier.

11. The therapeutic composition of claim 10, wherein the concentration of nanobubbles in the therapeutic composition is at least 10⁶ nanobubbles per cm³, at least 10⁷ nanobubbles per cm³, at least 10⁸ nanobubbles per cm³, at least 10⁹ nanobubbles per cm³, at least 10¹⁰ nanobubbles per cm³, or at least 10¹¹ nanobubbles per cm³.

12. The therapeutic composition of claim 10, wherein the antiparasitic agent is selected from the group consisting of hydrogen peroxide, organophosphates, pyrethroids, benzylureas, potassium ions, and combinations thereof.
